(19) United States
(12) Patent Application Publication
(10) Pub. No.: US 2017/0284954 A1
(43) Pub. Date: Oct. 5, 2017

(54) SYSTEMS AND METHODS FOR ELECTROCHEMICAL CREATININE ASSAYS

(71) Applicant: Polymer Technology Systems, Inc., Indianapolis, IN (US)

(72) Inventors: Gary L. Hughes, Camby, IN (US); Aniruddha Patwardhan, Fishers, IN (US); Christine Casterline, Carmel, IN (US)

(21) Appl. No.: 15/475,719

(22) Filed: Mar. 31, 2017

Related U.S. Application Data
(60) Provisional application No. 62/316,323, filed on Mar. 31, 2016.

(51) Int. Cl. G01N 27/327; C12Q 1/00

(52) U.S. Cl. CPC G01N 27/3272; G01N 27/3273; C12Q 1/005; C12Y 105/08003; C12Y 305/04021; C12Q 1/004; C12Y 105/03001; G01N 2333/9065; G01N 2333/978; G01N 2333/90683

(57) ABSTRACT

A system for the electrochemical detection of creatinine levels includes a test strip including an electrode and a counter electrode, the electrode and counter electrode located proximate to a sample reception area; and a coating on one of the electrode and counter electrode, the coating including a reagent coating for creatinine.

### CROSS-REFERENCE TO RELATED APPLICATIONS

This Application claims the benefit of Provisional Application No. 62/316,323 filed on Mar. 31, 2016, titled “Systems and Methods for Electrochemical Creatinine Assays,” the entire disclosure of which is hereby incorporated by reference.

### BACKGROUND

Creatine (C₄H₉O₂N₃ or α-methyl guanidine-acetic acid) is a compound present in vertebrate muscle tissue, principally as phosphocreatine. Creatine is synthesized primarily in the liver and also in the pancreas and the kidneys. Creatine helps produce energy needed to contract muscles, and it is produced at a relatively constant rate. Creatine eventually is spontaneously degraded into creatinine by muscle and is released into the blood. It then is excreted by the kidneys and removed by the body by glomerular filtration.

The amount of creatinine produced is relatively stable in a given person. Serum creatinine level, therefore, is determined by the rate it is being removed, which is roughly a measure of kidney function. If kidney function falls, serum creatinine levels will rise. Thus, blood levels of creatinine are a good measure of renal function. Usually, increased creatinine levels do not appear unless significant renal impairment exists.

According to the American Diabetes Association (ADA), 20% to 30% of patients with diabetes develop diabetic kidney disease (nephropathy). Further, some authorities recommend measurement of serum creatinine levels in non-diabetic patients to screen for renal dysfunction because of increasing evidence that dietary protein restriction and use of angiotensin-converting enzyme (ACE) inhibitors can retard progression once renal insufficiency develops. Thus, the need for creatinine testing as a measure of kidney function is well established.

### BRIEF SUMMARY

In one embodiment, a system for the electrochemical detection of creatinine levels includes a test strip including an electrode and a counter electrode, the electrode and counter electrode located proximate to a sample reception area; and a coating on one of the electrode and counter electrode, the coating including a reagent coating for creatinine. In one alternative, the reagent coating includes a surfactant, a binder, stabilizers, a buffer, sarcosine dehydrogenase, and potassium ferricyanide. In another alternative, the reagent coating includes sarcosine dehydrogenase and a mediator. Alternatively, the mediator is selected from the list consisting of methylene blue, meldora blue, phenazine methosulfate, 2,6-Diclorophenol indophenol, nile blue, and potassium ferricyanide. Optionally, the reagent coating includes a surfactant and a buffer. In one configuration, the reagent buffer includes a binder and a stabilizer. In another configuration, the reagent coating includes creatinine deiminase and a mediator. Optionally, the mediator is selected from the list consisting of methylene blue, meldora blue, phenazine methosulfate, 2,6-Diclorophenol indophenol, nile blue, and potassium ferricyanide. Alternatively, the reagent coating includes sarcosine oxidase and a mediator. In one configuration, the mediator is selected from the list consisting of methylene blue, meldora blue, phenazine methosulfate, 2,6-Diclorophenol indophenol, nile blue, and potassium ferricyanide.

In one embodiment, a system for the electrochemical detection of creatinine levels includes a test strip including an electrode and a counter electrode, the electrode and counter electrode located proximate to a sample reception area. The system further includes a coating on one of the electrode and counter electrode, the coating including a reagent coating for creatinine. The system further includes an analyzer for receiving the test strip and including instructions stored on a non-transitory medium for applying a current to the test strip and responsively determining an amount of creatinine. Optionally, the reagent coating includes sarcosine dehydrogenase and a mediator. Alternatively, the mediator is selected from the list consisting of methylene blue, meldora blue, phenazine methosulfate, 2,6-Diclorophenol indophenol, nile blue, and potassium ferricyanide. In one configuration, the reagent coating includes a surfactant and a buffer. In another configuration, the reagent buffer includes a binder and a stabilizer. Alternatively, the reagent coating includes creatinine deiminase and a mediator. Optionally, the mediator is selected from the list consisting of methylene blue, meldora blue, phenazine methosulfate, 2,6-Diclorophenol indophenol, nile blue, and potassium ferricyanide.

In one embodiment, a system for the electrochemical detection of creatinine levels includes a test strip including an electrode and a counter electrode, the electrode and counter electrode located proximate to a sample reception area. The system further includes a coating on one of the electrode and counter electrode, the coating including a reagent coating for creatinine. The system further includes an analyzer for receiving the test strip and including instructions stored on a non-transitory medium for determining a voltage of the test strip and responsively determining an amount of creatinine. Optionally, the reagent coating includes sarcosine dehydrogenase and a mediator. Alternatively, the mediator is selected from the list consisting of methylene blue, meldora blue, phenazine methosulfate, 2,6-Diclorophenol indophenol, nile blue, and potassium ferricyanide. In one configuration, the reagent coating includes creatinine deiminase and a mediator. In another configuration, the mediator is selected from the list consisting of methylene blue, meldora blue, phenazine methosulfate, 2,6-Diclorophenol indophenol, nile blue, and potassium ferricyanide. Optionally, a 1-methylhydantoinase (NMHase) and N-carbamoylsarcosine amidohydrolase (CSHase) enzyme may be used.

In one embodiment, a method of detecting creatinine includes providing an electrochemical test strip and placing the electrochemical test strip in an analyzer. The method further includes placing a blood sample or other biological fluid on the electrochemical test strip; measuring a current provided through the blood sample and the electrochemical test strip; and calculating a level of creatinine with the analyzer based on the current. Optionally, the test strip includes an electrode and a counter electrode, the electrode and counter electrode located in a sample reception area; and a coating on one of the electrode and counter electrode, the coating including a reagent coating for creatinine. Alternatively, the reagent coating includes sarcosine dehydrogenase and a mediator. In one alternative, the mediator is selected from the list consisting of methylene blue, meldora blue, phenazine methosulfate, 2,6-Diclorophenol indophenol, nile blue, and potassium ferricyanide. In another alternative, the reagent coating includes creatinine deiminase and a mediator. Optionally, the mediator is selected from the list consisting of methylene blue, meldora blue, phenazine methosulfate, 2,6-Diclorophenol indophenol, nile blue, and potassium ferricyanide.

### BRIEF DESCRIPTION OF THE DRAWINGS

FIG. 1 shows the proposed creatinine reagent scheme;

FIG. 2 shows typical creatinine/creatine detection reagent scheme;

FIG. 3 shows a proof-of-concept graph that was produced using whole blood;

FIG. 4 shows another proof-of-concept graph that was produced using creatinine in buffered solutions; and

FIG. 5 shows one embodiment of the strip design.

### DETAILED DESCRIPTION

Certain terminology is used herein for convenience only and is not to be taken as a limitation on the embodiments of the systems and methods for electrochemical creatinine assays. In the drawings, the same reference letters are employed for designating the same elements throughout the several figures.

To use a creatinine assay in the imaging market, a high level of precision is required to determine the difference between 1 and 1.1 mg/dL creatinine. This level of precision may be difficult to achieve with a reflectance-based test. Since electrochemical assays generally have better precision, it is desired to find such an approach for creatinine.

In some embodiments, an electrochemical reaction for a creatinine assay is proposed that significantly departs from present assays. The intended use may be to test whole blood or urine.

There are many advantages provided by an electrochemical creatinine assay, as compared to an optical assay. In contrast to many optical assays, by having an amperometric creatinine assay, no membranes are necessary. Many current optical assays are dependent on membrane manufacturers which discontinue membranes at their discretion.

Calibration of the analyzer may be easier with electrochemical testing. Measuring current (nA) is a standardized process, whereas standardizing reflectance is more difficult.

Testing electrochemically for creatinine may result in a cheaper cost per test strip due to less reagent, less raw materials (membranes, strip carriers, etc.), and automation of the process. Electrochemical test strips are generally inexpensive to produce due to the automation and small amounts of reagent used.

The proposed electrochemical creatinine assay is not dependent on oxygen and, thus, can test both venous and capillary blood.

Testing creatinine via electrochemistry will probably result in better precision. Precision and accuracy are key if this assay is to be developed for the imaging markets. Precision also will be aided by having four enzyme reactions instead of five.

The test range of an electrochemical creatinine assay may be larger than a reflectance assay in many embodiments. Reflectance tests are limited at the high concentrations by the amount of color that can be generated. However, electrochemical assays are able to measure much higher concentrations.

In some embodiments, the sample size will be small: ˜1.2 μL instead of 20 μL. In many embodiments, a transfer pipette is not needed to apply blood to a strip, since the blood sample simply is wicked into the sampling port.

Creatinine is a waste molecule from muscle metabolism. The bloodstream transports creatinine to the kidneys where the majority of it is filtered out and disposed as urine. Elevated creatinine levels are an indication of kidney malfunction. Creatinine is an important test to determine the functionality of the kidneys and can be used in the imaging markets to determine if contrast dye should be given to a patient.

There are several methods to measure creatinine. One popular chemical means is the Jaffe method, requiring no enzymes. The most popular enzymatic method is the reaction scheme listed below:

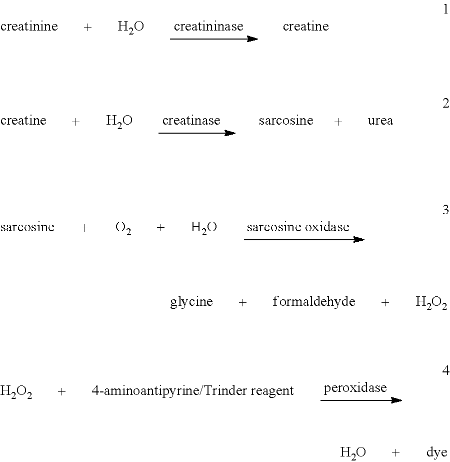

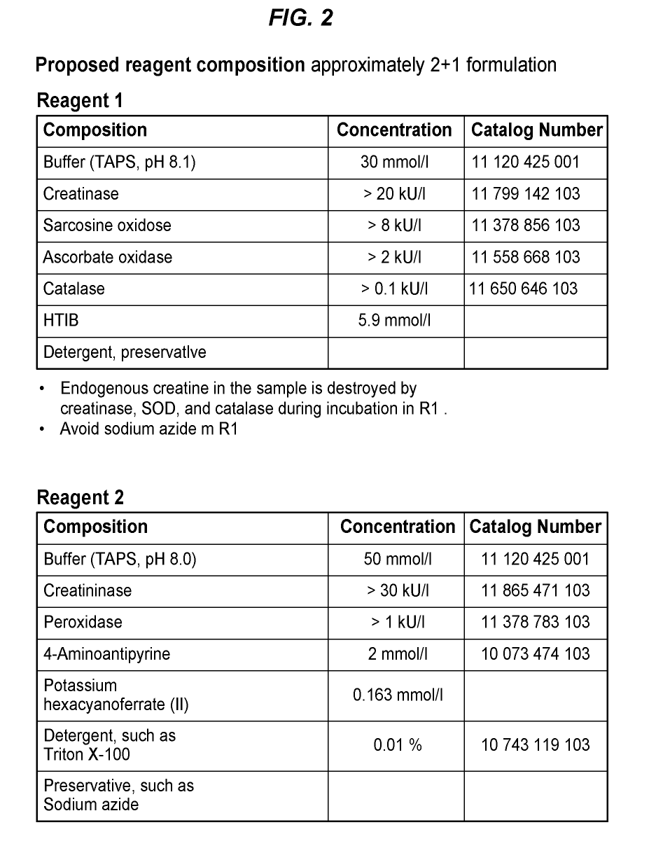

While this enzymatic method is a straightforward approach, it is not very suitable for point-of-care (POC) assays. Endogenous creatine will be an interferant, causing over recovery unless it is removed. Chemistry analyzers that use the above method are able to remove the creatine by reacting it with an enzyme train that results in no color formation for example the Roche's creatinine reagent (FIG. 2). FIG. 2 shows a creatinine reagent scheme. In order to use this method for a POC assay, there would have to be both a creatine assay and a creatinine assay with the creatine subtracted out in the final step. This doubles the cost of creating a POC creatinine assay, and there could be compounded errors based on the subtraction step.

In one embodiment, an improved creatinine assay was created. A more direct reaction scheme for a POC creatinine assay is listed in the equations below. This is a complex reaction with five different enzymes, taking approximately five minutes to test. It is also fairly expensive due to the enzyme costs in its optical form.

In some embodiments, this reaction is transformed into an electrochemical format to reduce the cost and time of the assay. One disadvantage of this pathway is that there still may be compounded errors from five enzyme reactions. Furthermore, the sarcosine oxidase is oxygen dependent. Having an electrochemical assay that is oxygen dependent is not desirable because of significant differences between venous and capillary blood. If sarcosine oxidase is replaced by sarcosine dehydrogenase, the oxygen interference is mitigated. Sarcosine dehydrogenase is rare but commercially available enzyme which has the following reaction:

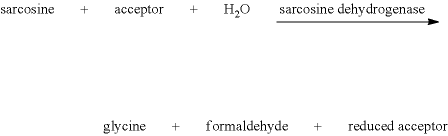

In many cases, the electron donor in a dehydrogenase reaction is nicotinamide adenine dinucleotide (NAD). However, NAD does not react well with sarcosine and sarcosine dehydrogenase. A list of potential electron acceptors was found from a journal article (see Imao Oka, Tadashi Yoshimoto, Kaoru Rikitake, Susumu Ogushi & Daisuke Tsuru, “Sarcosine Dehydrogenase from Pseudomonas putida: Purification and Some Properties,” Agricultural and Biological Chemistry, (1979) 43:6, 1197-1203), where methylene blue, meldora blue, phenazine methosulfate, 2,6-Diclorophenol indophenol, nile blue, and potassium ferricyanide were used. Interestingly, according to the journal article, potassium ferricyanide was only 90% efficient as methylene blue, meldora blue, phenazine methosulfate, or 2,6-Diclorophenol indophenol.

Based on the knowledge that ferricyanide could react in concert with sarcosine and sarcosine dehydrogenase, and because it was available, an electrochemical sarcosine sensor was created. In many embodiments, both electrochemical carbon and gold sensors were coated with reagent containing surfactant, binder, stabilizers, buffer, sarcosine dehydrogenase, and potassium ferricyanide. Solutions of sarcosine were made in a phosphate buffer at 40% hematocrit and tested on electrochemical test strips.

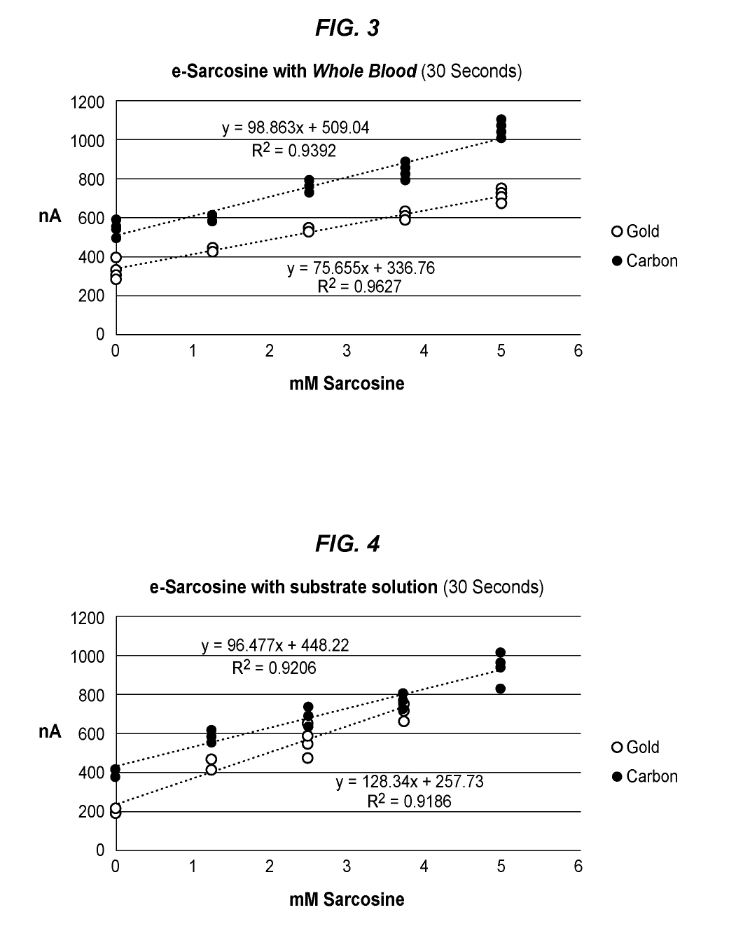

Valid results have been generated without optimization of any of the reagents. Optimization of pH, concentration of reactants, experimentation with mediators, etc., will yield better precision and greater slope for a creatinine assay. Finally, a mediator with lower reactivity was used in contrast to those thought to give more reactivity. Even with these limitations, the concept of an electrochemical creatinine assay according to the techniques described herein has been proven. FIG. 3 shows a proof-of-concept graph that was produced without any optimization of reagents. An electrochemical strip was made to test sarcosine solutions made with 40% hematocrit. Further optimization should allow for a lower intercept, better slope, and better precision.

FIG. 4 shows a proof-of-concept graph was produced without any optimization of reagents. The same strips in FIG. 3 were used to test solutions of sarcosine.

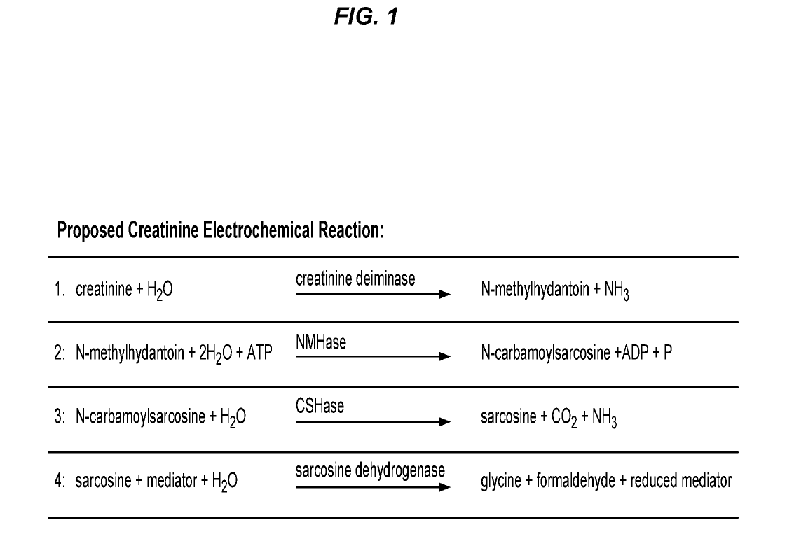

The equations in FIG. 1 show one embodiment of a proposed Creatinine Electrochemical Reaction.

At least one aspect of the pathway that was hypothesized and created was how the sarcosine would react with sarcosine dehydrogenase. As shown, a reaction with sarcosine, ferricyanide and sarcosine dehydrogenase have an electrochemical response on both gold and carbon sensors.

Embodiments of a system for detecting creatinine include an electrochemical creatinine assay by using sarcosine dehydrogenase coupled with a choice of mediators including the following: methylene blue, meldora blue, phenazine methosulfate, 2,6-Diclorophenol indophenol, nile blue, and potassium ferricyanide. A variety of other mediators may be used, including but not limited to combinations of the above mediators and a variety of other mediators. Potassium ferricyanide was chosen initially because we understand its properties. It appears from the referenced journal article that it probably will not be the mediator of choice.

Disclosed are embodiments of an electrochemical creatinine assay using the creatinine deiminase reaction pathway. Both the creatinine deiminase pathway and the creatininase pathway lead to the production of sarcosine. Should the creatinine deimnase reaction pathway be unsuitable due to performance, cost, etc., using the sarcosine dehydrogenase with a creatininase system would be a viable option, though not preferred.

Embodiments of the systems described herein have many advantages over other POC creatinine assays, including:
1. The chemical pathway utilizes four enzyme reactions instead of five;
2. Embodiments provide for a direct measurement of creatinine instead of testing creatinine and creatine and subtracting out the endogenous creatine;
3. Embodiments are oxygen independent allowing for venous or capillary blood to be used; and
4. Embodiments may be used to test either blood or urine.

In many embodiments, gold or carbon sensors (electrodes) may be used. Alternatively, platinum, silver chloride, or other types of electrodes may be used. An advantage of gold sensors is having less background signal while maintaining the same slope. Using gold sensors would also be advantageous for methods to measure hematocrit by AC impedance based on techniques that include the usage of phase angle shift in order to detect hematocrit.

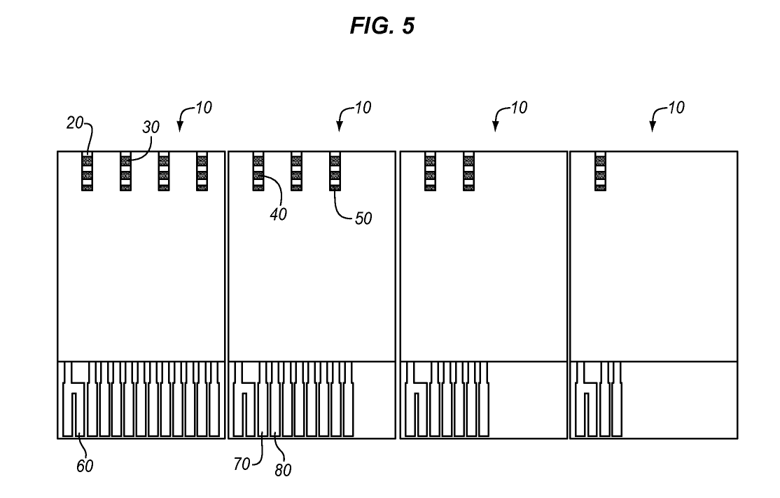

In addition to having an amperometric creatinine sensor, a versatile electrochemical test strip may offer multiple tests with the creatinine test. While the creatinine is tested, it may be helpful to check other important analytes such as glucose, ketones, triglycerides, etc. In some embodiments, an electrochemical sensor may include multiple testing areas as shown in FIG. 5.

FIG. 5 shows one embodiment of the strip design. Shown are four strips 10. From left to right, the strips 10 have 4, 3, 2, and 1 sample receiving ports 20. Each sample receiving port may have an electrode 30, a counter electrode 40, and a reference electrode 50. The reference electrode 50 may provide for a fill indication, as it will only pass a voltage when the sample reaches the electrode 50. The contacts 70, 80 also are visible, which interconnect with the electrodes and connect to contacts in the analyzer when inserted. The strip size does not change depending on the number of assays. In addition, the electrode placement does not change depending on the type of assays. Depending on what is desired for the testing scheme, sheets are printed for one, two, three, or four analytes. The spirit behind this invention disclosure is not to limit the size of the panel to only four analytes, but to provide a concept that is protected whether one or ten analytes are tested. Also, the electrodes do not all need to be on one side of the strip. Superior technology may be able to place electrodes on both sides of the strip, thus allowing for miniaturization.

In some embodiments, single analyte test strips are designed to have the same location with at least four associated electrodes. The electrode 60 that appears as an “h” is used for strip detection by the analyzer. The remaining assays will have at least three electrodes—one for sample fill detection, and the other two as a counter electrode and a working electrode. These assays are not limited to a set number of electrodes, for it is foreseen in some embodiments that more electrodes may be added for purposes of determining and correcting for hematocrit or other interfering substances.

In multiple configurations, reagents may be painted on the electrodes. Alternatively, reagents may be printed, coated, dip coated, or otherwise applied, as will be apparent in the field. Various types of electrodes may be used as well, including those made of carbon, gold, platinum, copper, or other conductive materials, as will be apparent to those in the field.

FIG. 5 displays separate blood sampling ports for each assay. Some embodiments may include separate sampling ports, particularly if there could be “cross talk” between reagents. In summary, embodiments of a novel idea for an electrochemical creatinine sensor have been presented. It is demonstrated that an electrochemical reaction with sarcosine, sarcosine dehydrogenase, and a mediator is a viable testing technique. An electrochemical creatinine test will have a smaller sample size, shorter test time, better precision, and will be cheaper to manufacture.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A system for the electrochemical detection of creatinine levels, the system comprising:
a test strip including an electrode and a counter electrode, the electrode and counter electrode located proximate to a sample reception area; and
a coating on one of the electrode and counter electrode, the coating including a reagent coating for creatinine.

2. The system of claim 1, wherein the reagent coating includes a surfactant, a binder, stabilizers, a buffer, sarcosine dehydrogenase, and potassium ferricyanide.

3. The system of claim 1, wherein the reagent coating includes sarcosine dehydrogenase and a mediator.

4. The system of claim 3, wherein the mediator is selected from the list consisting of methylene blue, meldora blue, phenazine methosulfate, 2,6-Diclorophenol indophenol, nile blue, and potassium ferricyanide.

5. The system of claim 4, wherein the reagent coating includes a surfactant and a buffer.

6. The system of claim 5, wherein the reagent buffer includes a binder and a stabilizer.

7. The system of claim 1, wherein the reagent coating includes creatinine deiminase and a mediator.

8. The system of claim 7, wherein the mediator is selected from the list consisting of methylene blue, meldora blue, phenazine methosulfate, 2,6-Diclorophenol indophenol, nile blue, and potassium ferricyanide.

9. The system of claim 1, wherein the reagent coating includes sarcosine oxidase and a mediator.

10. The system of claim 9, wherein the mediator is selected from the list consisting of methylene blue, meldora blue, phenazine methosulfate, 2,6-Diclorophenol indophenol, nile blue, and potassium ferricyanide.

11. A system for the electrochemical detection of creatinine levels, the system comprising:
a test strip including an electrode and a counter electrode, the electrode and counter electrode located proximate to a sample reception area;
a coating on one of the electrode and counter electrode, the coating including a reagent coating for creatinine; and
an analyzer for receiving the test strip and including instructions stored on a non-transitory medium for applying a current to the test strip and responsively determining an amount of creatinine.

12. The system of claim 11, wherein the reagent coating includes sarcosine dehydrogenase and a mediator.

13. The system of claim 12, wherein the mediator is selected from the list consisting of methylene blue, meldora blue, phenazine methosulfate, 2,6-Diclorophenol indophenol, nile blue, and potassium ferricyanide.

14. The system of claim 13, wherein the reagent coating includes a surfactant and a buffer.

15. The system of claim 14, wherein the reagent buffer includes a binder and a stabilizer.

16. The system of claim 11, wherein the reagent coating includes creatinine deiminase and a mediator.

17. The system of claim 16, wherein the mediator is selected from the list consisting of methylene blue, meldora blue, phenazine methosulfate, 2,6-Diclorophenol indophenol, nile blue, and potassium ferricyanide.

18. A system for the electrochemical detection of creatinine levels, the system comprising:
a test strip including an electrode and a counter electrode, the electrode and counter electrode located proximate to a sample reception area;
a coating on one of the electrode and counter electrode, the coating including a reagent coating for creatinine; and
an analyzer for receiving the test strip and including instructions stored on a non-transitory medium for determining a voltage of the test strip and responsively determining an amount of creatinine.

19. The system of claim 18, wherein the reagent coating includes sarcosine dehydrogenase and a mediator.

20. The system of claim 19, wherein the mediator is selected from the list consisting of methylene blue, meldora blue, phenazine methosulfate, 2,6-Diclorophenol indophenol, nile blue, and potassium ferricyanide.

21. The system of claim 18, wherein the reagent coating includes creatinine deiminase and a mediator.

22. The system of claim 21, wherein the mediator is selected from the list consisting of methylene blue, meldora blue, phenazine methosulfate, 2,6-Diclorophenol indophenol, nile blue, and potassium ferricyanide.

23. A method of detecting creatinine, the method comprising:
providing an electrochemical test strip;
placing the electrochemical test strip in an analyzer;
placing a blood sample on the electrochemical test strip;
measuring a current provided through the blood sample and the electrochemical test strip; and
calculating a level of creatinine with the analyzer based on the current.

24. The method of claim 18, wherein the test strip includes an electrode and a counter electrode, the electrode and counter electrode located in a sample reception area; and a coating on one of the electrode and counter electrode, the coating including a reagent coating for creatinine.

25. The method of claim 24, wherein the reagent coating includes sarcosine dehydrogenase and a mediator.

26. The method of claim 25, wherein the mediator is selected from the list consisting of methylene blue, meldora blue, phenazine methosulfate, 2,6-Diclorophenol indophenol, nile blue, and potassium ferricyanide.

27. The method of claim 24, wherein the reagent coating includes creatinine deiminase and a mediator.

28. The method of claim 27, wherein the mediator is selected from the list consisting of methylene blue, meldora blue, phenazine methosulfate, 2,6-Diclorophenol indophenol, nile blue, and potassium ferricyanide.
